(19) United States
(12) Patent Application Publication
(10) Pub. No.: US 2017/0199146 A1
(43) Pub. Date: Jul. 13, 2017

(54) PROSTATE CANCER SCREENING MODULE AND METHOD FOR OPERATING THE SAME

(71) Applicant: CHANG GUNG UNIVERSITY, TAOYUAN CITY (TW)

(72) Inventors: SIDDHESWAR MAIKAP, TAOYUAN CITY (TW); KANISHK SINGH, TAOYUAN CITY (TW)

(21) Appl. No.: 14/994,301

(22) Filed: Jan. 13, 2016

(51) Int. Cl. G01N 27/403; G01N 27/416; G01N 33/487; G01N 27/30

(52) U.S. Cl. CPC G01N 27/403; G01N 27/301; G01N 27/4161; G01N 33/48707; G01N 2333/90683

(57) ABSTRACT

The present invention relates to a prostate screening module and the method for operating the same. It uses nanometer-grade nickel oxide as the screening film. By contacting nickel oxide with hydrogen peroxide having various concentrations, the surface potential of nickel oxide will be changed and resulting in voltage shift, which can be used to judge the concentration of hydrogen peroxide. The sample for screening is urine or blood serum. After the sarcosine in urine or blood serum is oxidized and forming hydrogen peroxide, the content of sarcosine in the sample can be deduced by means of the reaction between hydrogen peroxide and nickel oxide. Accordingly, the possibility of prostate cancer can be evaluated.

### FIELD OF THE INVENTION

The present invention relates generally to a screening module and the method for operating the same, and particularly to a screening module capable of screening prostate cancer rapidly and the method for operating the same.

### BACKGROUND OF THE INVENTION

A prostate cancer comes from malignant tumors in the prostate. If gene mutation occurs and results in loss of control in proliferation, it becomes a cancer. In addition to expansion in volume or encroachment to neighboring organs, malignant cells can possibly transfer to other parts of the body, in particular bones and lymph nodes. A prostate cancer can lead to pains, impaired urinary elimination, or erection dysfunction.

A prostate cancer is mostly discovered during regular health examinations or blood screening. There are still doubts on the accuracy and effect of the prostatic specific antigen (PSA). Nonetheless, it remains the screening tool adopted most extensively for prostate cancers. In generally, when a suspect prostate cancer case is discovered, the biopsy is performed for validating a diagnosis. Other further examinations, such as X-ray scans, computerized tomography scans, and bond scans, can facilitate understanding if the prostate cancer has spread.

Unfortunately, the biopsy is after all an invasive examination. Besides, the examination is not immediate. Accordingly, in order to enhance the efficiency of screening prostate cancer, other methods should be sought as auxiliaries.

According to the current technology, scientists have found that sarcosine can be used as the biologic indicator for screening prostate cancer. Nonetheless, how to find the content of sarcosine in a sample rapidly and conveniently is still to be developed. The present invention provides a non-invasive method for screening rapidly and conveniently.

### SUMMARY

An objective of the present invention is to provide a prostate cancer screening module and the method for operating the same. By using the principle that the surface potential of the material of a sensing film (e.g. NiOₓ), varies by contacting hydrogen peroxide having different concentrations, the concentration of hydrogen peroxide can be judged by detecting voltage change. Then the content of sarcosine can be judged according to the concentration of hydrogen peroxide.

Another objective of the present invention is to provide a prostate cancer screening module and the method for operating the same. The sample under test is urine or blood serum. If the urine or blood serum contains sarcosine, hydrogen peroxide will be produced after oxidation. Then the produced hydrogen peroxide can be used to react with nickel oxide.

Still another objective of the present invention is to provide a prostate cancer screening module and the method for operating the same. Once the urine or blood serum sample contains traces of sarcosine, the voltage change owing to the change in the surface potential of the sensing film can be detected, leading to high sensitivity to sarcosine. The content of sarcosine in urine or blood serum is a factor for judging prostate cancer. Thereby, the present invention can be applied to screening prostate cancer.

A further objective of the present invention is to provide a prostate cancer screening module and the method for operating the same. The structure of the present invention is succinct and can be popularized as commercial screening chips. Thereby, screening for prostate cancer can become convenient and the result can be given immediately.

Accordingly, the present invention discloses a prostate cancer screening module, which comprises a conductive substrate, a p-type silicon semiconductor layer, a silicon dioxide layer, a sensing film, and a reference electrode. The p-type silicon semiconductor layer is disposed on the conductive substrate. The silicon dioxide layer is disposed on the p-type silicon semiconductor layer. The sensing film is disposed on the silicon dioxide layer for carrying a sample. The reference electrode is located above the sensing film for contacting the sample. Wherein the material of the sensing film is selected from the group consisting of NiOₓ, IrOₓ, GdOₓ, Pt, WOₓ, Si, Ge, Os, Pd, CrOₓ, CeOₓ, TaOₓ, ErOₓ, YOₓ, HfOₓ, ZrOₓ, SnOₓ, PrOₓ, SmOₓ, NbOₓ, ZnOₓ, LuOₓ, RuOₓ, MoS₂Oₓ, TmOₓ, HoOₓ, DyOₓ, YbOₓ, EuOₓ, TbOₓ, IGZOₓ, InNOₓ, NdOₓ, Al, Ti, and graphene oxide.

The method for operating the above prostate cancer screening module comprises steps of: using a reference electrode to approach a sensing film and giving a first voltage; disposing a sample between the reference electrode and the sensing film, giving a second voltage, and the sample being urine or blood serum; and comparing the first voltage and the second voltage for giving a difference value, and using the difference value to judge the content of sarcosine in the sample.

### BRIEF DESCRIPTION OF THE DRAWINGS

FIG. 1 shows a cross-sectional view of the prostate cancer screening module according to a preferred embodiment of the present invention;

FIG. 2 shows a flowchart according to a preferred embodiment of the present invention;

FIG. 3A and FIG. 3B show voltage test diagrams according to a preferred embodiment of the present invention for illustrating the normalized capacitance of titanium oxide film and nickel oxide film in various acid and alkaline ambiences, respectively;

FIG. 4 shows a voltage test diagram according to a preferred embodiment of the present invention for illustrating the sensitivities of films made of various materials; and

FIG. 5 shows a voltage test diagram according to a preferred embodiment of the present invention for illustrating the reactions of various materials with hydrogen peroxide.

### DETAILED DESCRIPTION

In order to make the structure and characteristics as well as the effectiveness of the present invention to be further understood and recognized, the detailed description of the present invention is provided as follows along with embodiments and accompanying figures.

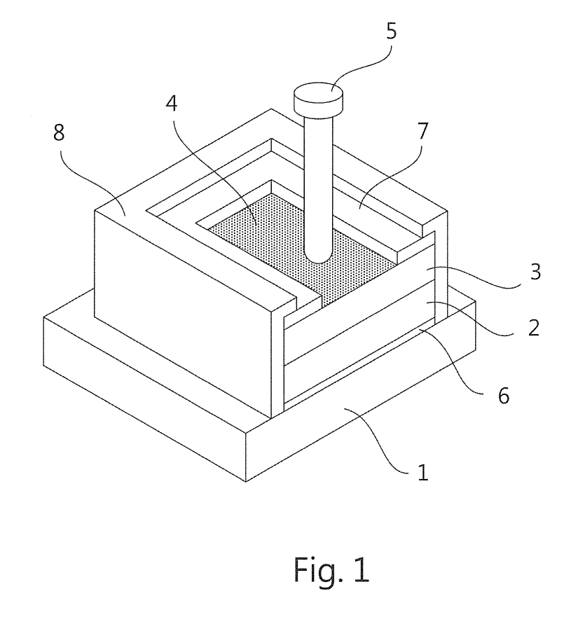

Please first refer to FIG. 1. A prostate cancer screening module according to a preferred embodiment of the present invention comprises a conductive substrate 1, a p-type silicon semiconductor layer 2, a silicon dioxide layer 3, a sensing film 4, and a reference electrode 5. The p-type silicon semiconductor layer 2 is disposed on the conductive substrate 1. The silicon dioxide layer 3 is disposed on the p-type silicon semiconductor layer 2. The sensing film 4 is disposed on the silicon dioxide layer 3. The reference electrode 5 is located above the sensing film 4. Wherein the material of the sensing film is selected from the group consisting of NiOₓ, IrOₓ, GdOₓ, Pt, WOₓ, Si, Ge, Os, Pd, CrOₓ, CeOₓ, TaOₓ, ErOₓ, YOₓ, HfOₓ, ZrOₓ, SnOₓ, PrOₓ, SmOₓ, NbOₓ, ZnOₓ, LuOₓ, RuOₓ, MoS₂Oₓ, TmOₓ, HoOₓ, DyOₓ, YbOₓ, EuOₓ, TbOₓ, IGZOₓ, InNOₓ, NdOₓ, Al, Ti, and graphene oxide. The selection of the material is based on the ability in changing the surface potential when contacted with hydrogen peroxide. Considering the cost, NiOₓ is preferred. Furthermore, because Si, Ge, Al and Ti are easy be oxidized in regular storage conditions, thus they are all potential in being the material of sensing films, however, additional cleaning process is needed.

In the structure according to the above preferred embodiment, the conductive substrate 1 is a copper-plated printed circuit board and can be used as an electrode corresponding to the reference electrode 5. The p-type silicon semiconductor layer 2 and the silicon dioxide layer 3 above the conductive substrate 1 enable the present invention to own the characteristics of an electrolyte-insulator-semiconductor sensor. The fabrication method for the structure is similar to normal semiconductor processes. By using chemical vapor deposition (CVD), plasma-enhanced CVD (PECVD), vapor deposition, e-gun vapor deposition, and radio-frequency (RF) sputtering, layers of different materials can be stacked. In order to increase electrical conductivity, according to a preferred embodiment, an aluminum electrode layer 6 is further included between the p-type silicon semiconductor layer 2 and the conductive substrate 1. Silver-silver chloride or other reference electrodes having a fixed potential difference can be selected to be the reference electrode 5.

According to a preferred embodiment, the sensing film 4 made by NiOₓ does not cover completely the silicon dioxide layer 3 in a thick film manner. Instead, nickel oxide is distributed on the silicon dioxide layer 4 in the form of nanometer particles. The thickness of the film is 1 to 5 nanometers and preferably 2 nanometers. According to another embodiment, titanium-oxide nanometer particles are further used to form a titanium oxide film. Nonetheless, the present invention is not limited to the use of titanium-oxide nanometer particles only to form the film. The titanium oxide film can be disposed below the sensing film 4. Alternatively, sputtering can be adopted to mix nickel-oxide nanometer particles and titanium-oxide nanometer particles and form the film. In other words, the titanium film and the sensing film 4 are disposed on the silicon dioxide layer 3 in a mixed fashion. According to still another preferred embodiment, after preparation of the titanium oxide film, a 650° C. annealing process can be performed for 30 minutes in ambient oxygen.

Please refer to FIG. 1 again. When the present invention is operating, the liquid sample is dripped on the surface of the sensing film 4. To prevent arbitrary flowing of excess sample, one or more resin block 7 can be further disposed on the silicon dioxide layer 3. This resin block 7 can partition and give a screening space above the silicon dioxide layer 3. The sensing film 4 as described above is located on the surface inside the screening space above the silicon dioxide layer 3. The material of the resin block 7 can be SU-8, which is normally used as negative photoresist. In the present invention, after spin coating, the material is baked and becomes the resin block 7 for partitioning.

In addition, the prostate cancer screening module can further comprise a housing 8 formed by epoxy resin. The housing 8 can protect the layers inside from pollution or oxidation and thus extending the lifetime of the prostate cancer screening module.

As described above, when the structure disclosed in the present invention is operating, the liquid sampled is dripped on the surface of the sensing film 4. According to a preferred embodiment of the present invention, the gradient of the sample is urine. Presently, it is known the content of sarcosine in urine is closely related to the screening of prostate cancer. Accordingly, the present invention makes use of chemical reactions to detect C-V (capacitance-to-voltage) changes and hence judging if the urine or blood serum contains sarcosine.

The sample used in the present invention is urine or blood serum. If the urine contains sarcosine, the following reaction will occur:

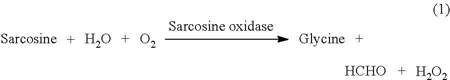

After oxidation, the sarcosine in urine produces hydrogen peroxide. Namely, if hydrogen peroxide exists in the sample, it implies existence of sarcosine in the urine. If the concentration of hydrogen peroxide is higher, then the content of sarcosine is greater, meaning that the characteristics of prostate cancer are more apparent. Sarcosine oxidase (SOD) can be added to the sample in the above oxidation reaction and acting as the catalyst. In order to make sure the existence and the concentrate of hydrogen peroxide, according to the present invention, the sample contacts the sensing film. When nickel oxide contacts hydrogen peroxide, the following reduction reactions occur:

NiO+OH⁻→NiOOH (2)

2NiOOH+H₂O₂→2NiO+2H₂O+O₂ (3)

In Formula (2), nickel oxide produces nickel oxyhydroxide in alkaline ambience. Then, in Formula (3), the oxyhydroxide further reacts with hydrogen peroxide and is reduced to nickel oxide. The oxidation number of nickel is changed from +2 to +3. Besides, the potential is changed and thereby the screening module as described above can be used to detect changes in voltage. Consequently, the content of sarcosine in urine can be judged sensitively and real-timely for evaluating if a prostate cancer occurs.

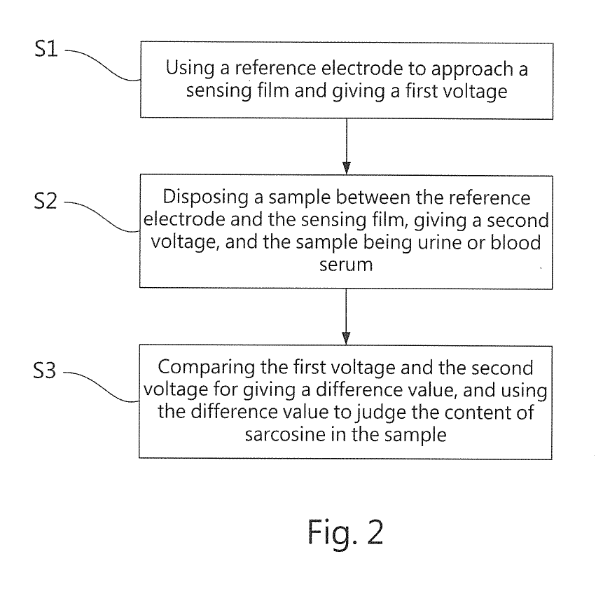

Please refer to FIG. 2. Based on the principle and structure disclosed above and according to a preferred embodiment of the present invention, the method for operating the prostate cancer screening module comprises steps of:
Step S1: Using a reference electrode to approach a sensing film and giving a first voltage;
Step S2: Disposing a sample between the reference electrode and the sensing film, giving a second voltage, and the sample being urine or blood serum; and
Step S3: Comparing the first voltage and the second voltage for giving a difference value, and using the difference value to judge the content of sarcosine in the sample.

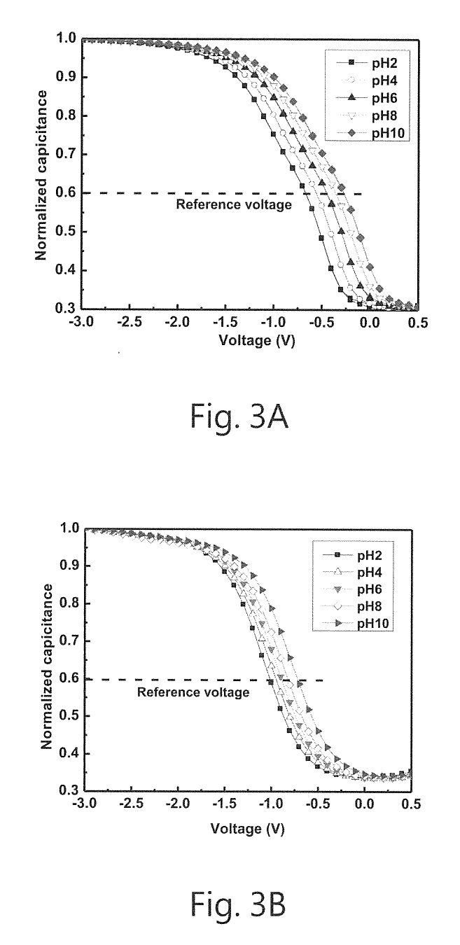

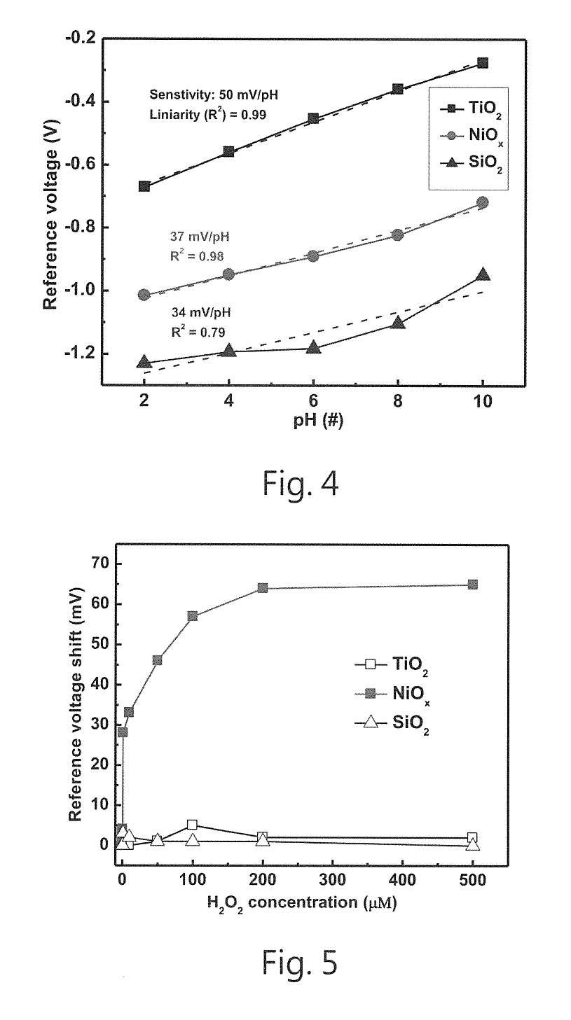

Please refer to FIGS. 3A and 3B, which illustrate the sensitivity behaviors of the nickel oxide film and the titanium oxide film disposed on the silicon dioxide layer of the screening module in different acid and alkaline ambiences disclosed in the present invention. In the test process, the thicknesses of the nickel oxide film and the titanium oxide film are both 2 nanometers and the screening module is immersed in a buffer solution with pH values ranging from 2 to 10. As shown in the figures, different acid and alkaline ambience result in changes of the measured reference voltage. This is caused by the changes in the surface potential of the sensing films, namely, the nickel oxide film and the titanium oxide film. By C-V measurements, these changes can be detected. Please further refer to FIG. 4 for comparison. It is known from the figure that the titanium oxide film has a better acid and alkaline sensitivity of 50 mV/pH with a linearity of 0.99. On the other hand, nickel oxide film has a sensitivity of 37 mV/pH with a linearity of 0.98, and silicon dioxide film has a sensitivity of only 34 mV/pH with a linearity of 0.79. Accordingly, in the previous preferred embodiment of the present invention, titanium oxide is mixed (SiO₂ and NiOₓ are also available but TiOₓ is preferred) into the sensing film. The purpose is just to improve the sensitivity of detection.

Please refer to FIG. 5, which shows detection results of the sensing film on hydrogen peroxide by using various materials as the sensing film. The test is performed in a 10 mM PBS buffer solution with an ambient pH value of 7. As shown in the results, in practice, the one having apparent reaction with hydrogen peroxide is the nickel oxide film. Contrarily, titanium oxide and silicon dioxide materials demonstrate no activity with hydrogen peroxide. Accordingly, the present invention adopts nickel oxide as the major material of the sensing film.

Besides, while applying the present invention, a buffer solution can be further added between the sensing film and the reference electrode. The function of this buffer solution is to influence the pH value of the sample and thus adjusting the substrate bias.

To sum up, the present invention discloses in detail a prostate cancer screening module and the method for operating the same. The sample for detection is urine or blood serum. If the urine or blood serum contains elevated level of sarcosine, which is relevant to the prostate cancer, the sarcosine can be oxidized to produce hydrogen peroxide by reacting with an enzyme sarcosine oxidase. Then a sensing film made of nickel oxide can be used to react with the hydrogen peroxide and changing the surface of the sensing film. By detecting the voltage changes, the concentration of the hydrogen peroxide can be judged and hence deducing the content of sarcosine in the sample. The present invention owns the feature of rapid screening and high sensitivity. Accordingly, it is undoubtedly an extremely valuable prostate cancer screening module and the method for operating the same.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

What is claimed is:

1. A prostate cancer screening module, comprising:
a conductive substrate;
a p-type silicon semiconductor layer, disposed on said conductive substrate;
a silicon dioxide layer, disposed on said p-type silicon semiconductor layer;
a sensing film, disposed on said silicon dioxide layer for carrying a sample; and
a reference electrode, located above said sensing film for contacting said sample;
wherein the material of the sensing film is selected from the group consisting of NiOₓ, IrOₓ, GdOₓ, Pt, WOₓ, Si, Ge, Os, Pd, CrOₓ, CeOₓ, TaOₓ, ErOₓ, YOₓ, HfOₓ, ZrOₓ, SnOₓ, PrOₓ, SmOₓ, NbOₓ, ZnOₓ, LuOₓ, RuOₓ, MoS₂Oₓ, TmOₓ, HoOₓ, DyOₓ, YbOₓ, EuOₓ, TbOₓ, IGZOₓ, InNOₓ, NdOₓ, Al, Ti, and graphene oxide.

2. The prostate cancer screening module of claim 1, wherein said conductive substrate is a copper-plated printed circuit board.

3. The prostate cancer screening module of claim 1, wherein an aluminum electrode layer is disposed between said p-type silicon semiconductor layer and said conductive substrate.

4. The prostate cancer screening module of claim 1, wherein the thickness of said sensing film is between 1 and 2 nanometers.

5. The prostate cancer screening module of claim 1, and further comprising one or more resin block on said silicon dioxide layer for partitioning and giving a screening space, and said sensing film located on said silicon dioxide layer in said screening space.

6. The prostate cancer screening module of claim 1, wherein said sample is urine or blood serum.

7. The prostate cancer screening module of claim 1, and further comprising a titanium film disposed below said sensing film.

8. The prostate cancer screening module of claim 1, and further comprising a titanium film mixed with said sensing film and disposed on silicon dioxide layer.

9. A method for operating a prostate cancer screening module, comprising steps of:
using a reference electrode to approach a sensing film and giving a first voltage;
disposing a sample between said reference electrode and said sensing film, giving a second voltage, and said sample being urine or blood serum; and
comparing said first voltage and said second voltage for giving a difference value, and using said difference value to judge the content of sarcosine in said sample;
wherein the material of the sensing film is selected from the group consisting of NiOₓ, IrOₓ, GdOₓ, Pt, WOₓ, Si, Ge, Os, Pd, CrOₓ, CeOₓ, TaOₓ, ErOₓ, YOₓ, HfOₓ, ZrOₓ, SnOₓ, PrOₓ, SmOₓ, NbOₓ, ZnOₓ, LuOₓ, RuOₓ, MoS₂Oₓ, TmOₓ, HoOₓ, DyOₓ, YbOₓ, EuOₓ, TbOₓ, IGZOₓ, InNOₓ, NdOₓ, Al, Ti, and graphene oxide.

10. The method for operating a prostate cancer screening module of claim 9, wherein said sample contains a sarcosine oxidase.
